(19) United States
(12) Patent Application Publication
(10) Pub. No.: US 2013/0156739 A1
(43) Pub. Date: Jun. 20, 2013

(54) PRODUCT FOR THE UPPER GASTRIC SPHERE

(71) Applicants: Ricardo Julian Weill, Capa (AR); Céline Valentini, Chatillon (FR)

(72) Inventors: Ricardo Julian Weill, Capa (AR); Céline Valentini, Chatillon (FR)

(73) Assignees: DANONE ARGENTINA S.A., Buenos Aires (AR); COMPAGNIE GERVAIS DANONE, Paris (FR)

(21) Appl. No.: 13/814,887

(22) PCT Filed: Aug. 9, 2011
(86) PCT No.: PCT/EP11/63697

§ 371 Date: Feb. 7, 2013

(30) Foreign Application Priority Data
Aug. 13, 2010 (IB) PCT/IB2010/002436

(51) Int. Cl. A61K 33/08; A61K 31/194; A61K 31/045; A23L 1/29; A61K 33/06

(52) U.S. Cl. CPC A61K 33/08; A23L 1/296; A61K 33/06; A61K 31/045; A61K 31/194
USPC 424/93.4; 424/692

(57) ABSTRACT

The invention concerns a product for the upper gastric sphere of humans. The product can address acidity in upper gastric sphere of humans. The product of the invention is typically to be administered orally, typically as food or food supplement. The product comprises a base product and several agents that provide benefits to the upper gastric sphere.

The invention concerns a product for the upper gastric sphere of humans, in particular a product that can address acidity in upper gastric sphere of humans. The product of the invention is typically to be administered orally, for example as food or food supplement. The product comprises a base product and several agents that provide benefits to the upper gastric sphere.

### BACKGROUND OF THE INVENTION

Some studies showed that the presence of acidity in the upper part of the digestive system (also referred to as upper gastric sphere) is associated to a low quality of life. A large number of people need relief from acidity, or from the sensation of acidity, because it provides discomfort but also because it requires a high level of medical resources and/or expenses for its treatment.

The sensation of acidity is one symptom of two chronic disorders: Gastro-oesophageal reflux disease (GERD) and functional dyspepsia.

Gastro-Esophageal Reflux Disease (GERD), that affects 5 to 10% of the population, is defined as a condition which appears when the reflux of gastric content in the esophagus causes troublesome symptoms and/or complications. GERD syndromes are typically divided into oesophageal and extra-oesophageal syndromes, both detailed below.

GERD oesophageal syndromes: Symptomatic syndromes localized in the esophagus are chest pain syndrome and two typical reflux syndromes: The first reflux syndrome is pyrosis. This cardinal syndrome of GERD is observed in 10-48% of the general population. It is defined has heartburn or burning sensation rising up from the stomach behind the chest. It is considered recurrent pyrosis when it happens at least twice a week. The second reflux syndrome is regurgitation, defined by stomach contents rinsing up to throat or mouth without nauseas or vomiting. This might lead to esophageal injuries like esophagitis, stricture, Barrett.

GERD Extra-esophageal syndromes: Symptomatic syndromes include reflux associated cough, laryngitis, asthma and dental erosions.

GERD is considered as pathologic when there are chronicle symptoms and mucosal damage.

Functional dyspepsia, which affects about 25% of the population, is defined as a sensation of pain or discomfort located in the upper abdomen according to ROME II criterion. It is identified by one or more of the following bothersome: postprandial fullness, early satiety, epigastric pain and epigastric burning.

Patients suffering from dyspepsia should be distinguished from those reporting predominant retrosternal burning (heartburn), who are very likely to suffer from GERD. However GERD may be accompanied with some dyspeptic symptoms, which leads to an overlap between GERD and dyspepsia.

Although incompletely understood, it is clear that the pathophysiology of GERD is multifactorial. The pressure of the lower oesophageal sphincter, the motility of the oesophageal body and the stomach, the composition of the reflux material, and the sensitivity or resistance of the oesophageal mucosa to the reflux material, are important factors involved in the pathogenesis of GERD-related symptoms and lesions. The refluxate is not only composed of gastric juice (acid and pepsin), but may also contain food and regurgitated duodenal contents. In adults, reflux of duodenal contents into the stomach is a physiological event, especially in the postprandial period and during the night. Hence, it is not unusual that the refluxate arising from the stomach contained duodenal contents, this syndrome being referred to as duodeno-gastro-oesophageal reflux (DGER).

Therefore, the refluxate can contain both bile and pancreatic enzymes in addition to acids and pepsin. The compounds of the refluxate can cause lesions of the oesophageal mucosa and heartburn. Hydrochloric acid present in the gastric refluxate indeed exerts cell-damaging effects by disturbing the pH/ion balance. However, in vitro, in vivo and human studies lead to the conclusions that oesophageal mucosa is relatively resistant to acid exposure (alone), and that it is the addition of pepsin and/or bile salts that enhances tissue damages. Indeed, proteolytic enzymes such as pepsin and trypsin have the capacity to disrupt epithelial structures by digesting cell surfaces and promoting cell shredding. In particular, pepsin is thought to be the major cause of heartburn and oesophagitis when associated to an acid, as its enzymatic activity is optimal when the pH is significantly low. Acid and pepsin can bring about direct cellular damage and also disruption of intracellular junctions.

Bile salts, formed in the liver by oxidation of cholesterol and present in bile at high concentrations, are critically important for absorption of lipids from the intestine. They can disrupt the integrity of the oesophageal mucosa via a detergent effect on the cell membranes and on the cell-to-cell adhesions. Alternatively, their lipophilic state can allow them to cross the mucosa and disorganize membrane structure or interfere with cellular function.

According to a work carried out in Argentina on a 1000-surveyed population, GERD, defined as the presence of pyrosis or regurgitation (also sometimes referred to as “acidity episodes”) once or several times a week affected an average of 23.0% of the population. In general, pyrosis in patients may be diagnosed through anamnesis and does not require further studies. However, the presence of abnormal or extra oesophageal symptoms of GERD, such as chronic cough, laryngitis, pharyngeal or balloon sensation among others, renders the diagnosis determination difficult.

In order to evaluate common and abnormal symptoms of GERD and to quantify its impact on the quality of life, questionnaires validated for language have been prepared, for example, Reflux Disease Questionnaire (RDQ), Gastrointestinal Impact Scale (GIS) and QOLRAD.

Indeed, there is a clear need to address acidity (including the sensation of acidity, pyrosis, and heartburn), and/or to provide other benefits to the upper gastric sphere. For quality of life reasons, these symptoms are really affecting day to day life, but also for prevention of complication like oesophageal injuries.

Treatment of symptomatic GERD based on antacid solutions containing magnesium (Mg), Calcium (Ca), Aluminium (Al) salts and/or alginates is widely used with or without medical prescription. Such treatments include administration of antacid compositions that increase the pH of the stomach juice, thus increasing the pH of the reflux in the oesophagus which results in a decreased of the acidity sensation.

One way of improving oesophageal mucosa protection against luminal noxa is the consumption of viscous products that will provide an adherent layer over the mucosa.

Antacid products are commercially available pharmaceutical products, either with a prescription or over-the-counter. These products, however, have a very high antacid activity and generally increase stomach pH, typically over pH=5. Such values do not allow the concomitant administration of some other pharmaceutical products. Meanwhile pHs higher than pH=5 in the stomach deactivate some of the stomach enzymes, thus disturbing the digestion process. Hence, these products should be administered during a limited period of time, as confirmed by the instructions provided with such products. Also, these products might require specific medical attention. They cannot be used on a long-term period to regulate and/or prevent acidity troubles. In addition, their action depends on the initial pH and/or on the initial volume of the stomach on which they are administered. There is a need for products providing a softer or more controlled (for example with less variations) antacid action and/or a more consistent action.

### SUMMARY OF THE INVENTION

The invention addresses at least one of above-mentioned needs and/or one of the above-mentioned problems with a product comprising:

a) optionally a salivation agent,
b) optionally a mouth and/or throat cooling agent,
c) at least one gum,
d) a phospholipid product,
e) a base product comprising a mineral salt with a base anion, preferably a magnesium, sodium, calcium, or aluminum salt, and
f) a solid or liquid matrix.

The product can typically be a food product, a food supplement product or a pharmaceutical product.

The invention also concerns a process for preparing said product.

The invention also concerns the product for use in:

providing stomach and/or gastric comfort and/or health,

providing prevention of, relief from, and/or treatment of stomach and/or esophagus pain,

providing prevention of, relief from, and/or treatment of acidity or acidity sensation in the upper gastric sphere of humans, preferably in stomach and/or esophagus,

providing prevention of, relief to, and/or treatment of pyrosis and/or heartburn, and/or

providing prevention of, relief to, and/or treatment of Gastro-Esophageal Reflux Disease (GERD) and/dyspepsia.

The invention also concerns the use of the product for:

providing stomach and/or gastric comfort and/or health,

providing prevention of, relief from, and/or treatment of stomach and/or esophagus pain,

providing prevention of, relief from, and/or treatment of acidity or acidity sensation in the upper gastric sphere of humans, preferably in stomach and/or esophagus,

providing prevention of, relief to, and/or treatment of pyrosis and/or heartburn, and/or

providing prevention of, relief to, and/or treatment of Gastro-Esophageal Reflux Disease (GERD) and/dyspepsia.

The invention also concerns a method of:

providing stomach and/or gastric comfort and/or health,

providing prevention of, relief from, and/or treatment of stomach and/or esophagus pain,

providing prevention of, relief from, and/or treatment of acidity or acidity sensation in the upper gastric sphere of humans, preferably in stomach and/or esophagus,

providing prevention of, relief to, and/or treatment of pyrosis and/or heartburn, and/or

providing prevention of, relief to, and/or treatment of Gastro-Esophageal Reflux Disease (GERD) and/dyspepsia,

said method comprising the step of administrating the product, preferably orally.

The invention also concerns the use of the product for the manufacture of a health-related product (typically a medicine or a functional food or food supplement) to be used in:

providing stomach and/or gastric comfort and/or health,

providing prevention of, relief from, and/or treatment of stomach and/or esophagus pain,

providing prevention of, relief from, and/or treatment of acidity or acidity sensation in the upper gastric sphere of humans, preferably in stomach and/or esophagus,

providing prevention of, relief to, and/or treatment of pyrosis and/or heartburn, and/or

providing prevention of, relief to, and/or treatment of Gastro-Esophageal Reflux Disease (GERD) and/dyspepsia.

### DETAILLED DESCRIPTION OF THE INVENTION

### Definitions

In the present specification, amounts are expressed as active amounts (as opposed to amounts of ingredients “as is”) unless otherwise specified.

In the present specification, a matrix refers to a compound or a composition of matter wherein some ingredients (at least optionally a), optionally b), c), d), e), and ingredients described as further ingredients) are dispersed, for example solubilised, supported, suspended, embedded, emulsified. All the water present in the product of the invention is considered as being a part of the matrix. Compounds present in milks (animal or vegetal), or fruit juices, or fermentation products thereof, are also considered as being part of the matrix.

In the present specification, the term “use” of a compound or a composition is intended to cover the use itself, optionally including the intention to use, but also encompasses any commercial or legal communication associated to the compound(s) or to the compositions, for example advertisement, instructions or recommendation on the packages of the compositions, instructions or recommendation on any commercial supports such as leaflets, brochures, posters, websites, documentations filed in support to regulatory registrations for safety purpose, efficacy purpose, or consumer protection, for example to administrations such as EFSA in Europe and FDA in USA.

In the present application, the term “upper gastric sphere” refers to the part of the digestive system that comprises the stomach, the esophagus, and the mouth.

In the present specification the term “or” is not exclusive.

The product

The product of the invention comprises the following ingredients:

a) optionally a salivation agent,
b) optionally a mouth and/or throat cooling agent,
c) at least one gum,
d) a phospholipid product,
e) a base product comprising a mineral salt with a base anion, preferably a magnesium, sodium, calcium, or aluminum salt, and
f) a solid or liquid matrix.

The product can, for example, be a food product, a food supplement or a pharmaceutical product. The product can be used and made available to users as a consumer food available in food stores (including web-sites), as food supplements available in food stores or in specialized retail networks (or web sites), as a prescription pharmaceutical product available in pharmacies or as an over-the-counter pharmaceutical product available in pharmacies or via other networks (including web-sites).

In a preferred embodiment, the product is a food product, such as a dairy product, (preferably available in chilled shelves at a temperature ranging from 0° C. to 10° C.), or such as frozen products.

The association of ingredients optionally a), optionally b), c), d), e) and f) in the product of the invention provides a good relief from acidity and/or from acidity sensation. It helps people feeling better, and it reduces acidity (including the sensation of acidity) or at least the discomfort associated to acidity episodes.

It is believed that such effect is linked to a sensation in the mouth and/or in the throat associated with the absorption of the product, including salivation and/or cooling and/or viscous texture and/or pseudoplastic texture, and to the actual effect in the esophagus and in the stomach (acidity management and/or protection of the mucosa). It has been found that the association of parts or all of the ingredients provides a synergy on the viscosity.

Some further details or ingredients are detailed below.

### a) Salivation Agent

The product of the invention optionally comprises at least one salivation agent. Salivation agents are agents that can promote the production of saliva. Such agents are known by the one skilled in the art and have been documented. These agents are also referred to as mouth watering agents, mouth wetting agents or mouth hydrating agents.

The production of saliva is stimulated by both the sympathetic and the parasympathetic nervous system. The saliva stimulated by sympathetic innervation is thicker whereas the saliva stimulated parasympathetically is more watery. Parasympathetic stimulation leads to acetylcholine (ACh) release onto the salivary acinar cells. ACh binds to muscarinic receptors and causes an increased intracellular calcium ion concentration (through the IP₃/DAG second messenger system). Increased calcium causes vesicles within the cells to fuse with the apical cell membrane leading to secretion formation. ACh also causes the salivary gland to release kallikrein, an enzyme that converts kininogen to lysyl-bradykinin. Lysyl-bradykinin acts upons blood vessels and capillaries of the salivary gland to generate vasodilation and increased capillary permeability respectively. The resulting increased blood flow to the acinar allows production of more saliva. Lastly, both parasympathetic and sympathetic nervous stimulation can lead to myoepitheilium contraction which causes the expulsion of secretions from the secretory acinus into the ducts and eventually to the oral cavity.

Produced in salivary glands, human saliva is typically constituted of about 98% water, but it contains many other substances, including electrolytes, mucus, antibacterial compounds, cells, various enzymes.

The pH of the saliva normally varies (alkalinity vs. acidity) between 6.2 and 7.4, with higher pH levels often observed when secretion of saliva increases, for instance when smelling food odors or when watching pictures of food while hungry.

The salivation agent can for example be a mono-, di-, or tricarboxylic acid or a salt thereof, preferably succinic acid or a salt thereof. According to the invention, a carboxylic acid is a compound comprising at least a carboxylic group. It might also comprise other groups such as hydroxyl groups. Hydroxycarboxylic acids are thus encompassed.

Examples of suitable dicarboxylic acids include oxalic acid, malonic acid, succinic acid, glutaric acid, adipic acid, pimalic acid, octanedioic acid, nonanedioic acid, decandioic acid, undecanedioic acid, dodecanedioic acid, tridecan dioic acid, tetradecanedioic acid, pentadecane dioic acid, hexadecanedioic acid, octadecanedioic acid, fumaric acid, malic acid, tartaric acid. Examples of suitable tricarboxylic acids include citric acid. Other examples of suitable salivation agents include ascorbic acid. Suitable salts of such acids include sodium salts, potassium salts and calcium salts. Folic acid presents two carbolic acid groups, but is preferably not used. Citric acid or salts thereof such as potassium citrate is not preferred. If potassium citrate is used, it is preferably associated with a cooling agent. Ascorbic acid or salts thereof is not preferred. If ascorbic acid is used, it is preferably associated with a cooling agent. If folic acid is used, it is preferably associated with a cooling agent.

In one embodiment of the invention, the salivation agent a) is a succinic acid or a salt thereof such as potassium succinate.

The product of the invention can typically comprise from 0.0001% to 0.1% by weight, preferably from 0.001% to 0.05%, preferably from 0.001 to 0.01% of salivation agent.

The salivation agent can be introduced in the product of the invention in the form of a composition of matter comprising the salivation agent, optionally a cooling agent, and optionally flavors. In some cases, the composition of matter comprises water and/or a solvent such as ethanol as medium.

It is believed that the salivation agent contributes in diluting the stomach acidity and/or in increasing the pH of the stomach. The salivation agent can also help in providing relief and/or refreshing sensation.

b) Mouth and/or Throat Cooling Agent

The product of the invention optionally comprises a mouth and/or throat cooling agent.

By “cooling agent”, it is to be understood, in the meaning of the present invention, any agent that imparts a cooling sensation to the skin and mucous membranes of the body, particularly the mouth, nose, throat and gastrointestinal tract during its consumption. Cooling agents are known in the art, for example from U.S. Pat. No. 7,090,832. The cooling agent may be selected from menthol and menthol derivative compounds, acyclic and/or cyclic carboxamides, N-substituted paramenthane carboxamides, phosphine oxides, substituted p-menthanes, menthoxypropane, alpha-keto enamine derivatives, N-substituted p-menthane carboxamide, menthyl half acid ester derivatives, cubebol etc. Preferably, the cooling agent is a mixture of compounds comprising at least a menthol compound and a cyclic carboxamide compound.

The product of the invention can typically comprise from 0.0001% to 0.1% by weight, preferably from 0.001% to 0.05%, preferably from 0.001 to 0.01% of cooling agent.

The cooling agent can be introduced in the product in the form of a composition of matter comprising the cooling agent, optionally a salivating agent, and optionally flavors. In some cases the composition of matter comprises water and/or a solvent such as ethanol as a medium.

The cooling agent can also contribute in providing relief and/or a refreshing sensation.

### c) Gum

The product of the invention comprises at least one gum. Gums herein are understood to mean a polysaccharide polymers typically obtained from plants or microbiological processes. Some gums can be obtained by a process including a further step of chemical modification, for example grafting or modifying some of the side groups. Such products and processes are known from the one skilled in the art. Gums are typically used in food products as thickening agents to gain viscosity. One can use mixtures of gums.

The gum is preferably chosen so that the product of the invention shows pseudoplasticity, or so that it develops pseudoplasticity upon addition to water. The product can have pseudoplasticity as such, typically when it is in a liquid form, for example when comprising a liquid matrix. When the product is in a form suitable for dilution in water, for example in a solid form, typically with a solid matrix, the pseudoplasticity can be obtained after dilution in water. Addition or dilution of the gum in water can for example be at a 10% by weight concentration. The nature of the gum (or mixture of gums) and/or its amount can be set in order to obtain such pseudoplasticity.

The gum is also preferably chosen such that the product presents viscosity recovery. The nature of the gum (or mixture of gums) and/or its amount can be set to obtain said viscosity recovery.

Viscosity recovery is obtained when at least 90% of the initial viscosity (measured at a shear of 10 s⁻¹) is recovered within 1 s (preferably less than 1/10 s) after having increased the shear from an initial value of 10 s⁻¹ to 50 s⁻¹ or 100 s⁻¹, and then decreased it back to 10 s⁻¹.
Viscosity recovery is preferably obtained when at least 95%, and more preferably 99% of the initial viscosity (measured at a shear of 10 s⁻¹) is recovered within 1 s (preferably less than 1/10 s) after having increased the shear from an initial value of 10 s⁻¹ to 50 s⁻¹ or 100 s⁻¹, and then decreased it back to 10 s⁻¹.
It is believed that viscosity recovery can help in efficiently protecting the esophagus, and/or in providing relief, as it helps the product to well cover the mucosa and/or provides a positive sensory effect.

Gums with high molecular weight are especially suitable.

The gum can be for example be selected form the group consisting of the following compounds:

Gellan gums,
xanthan gums,
maltlodextrins,
locust bean gums,
carraghenan gums,
alginates,
carboxymethylcelluloses,
methylcelluloses,
galactomannans such as guar, and
mixtures thereof.

It is mentioned that some gums may be used as salts versions (for example sodium alginate, sodium carboxymethylcellulose).

Xanthan gum is typically obtained by biofermentation using a sugar source. It is typically used in food products as a thickening agent or as a stabilizing agent. Xanthan Gums are typically produced by a fermentation process using the bacteria Xanthomonas campestris. The composition and structure of xanthan gum produced by commercial fermentation is identical to the naturally occurring polysaccharide formed by Xanthomonas campestris on plants belonging to the cabbage family.

Xanthan gums typically have high viscosities at low shear rates, which can stabilize suspensions, yet provide good flow properties when poured or spooned from a container. At the processing stage, low viscosity at high-shear rates makes xanthan gum solutions easily pumped and poured. Xanthan gum is characterized by its very high viscosity at low concentrations. Because of its pseudoplastic property, it imparts excellent stability to oil-in-water emulsions by preventing the oil droplets from coalescing. Food grade 200 meshes xanthan gums with a molecular weight from 1000000 g/mol to 2000000 g/mol are especially suitable.

Gellan gum is typically obtained by Biofermentation using a sugar source. It is typically used in food products as a gelling agent, as a texturing agent, as a suspending agent or as a film forming agent. Gellan gums are typically produced by fermentation of a pure culture of Sphingomonas elodea. The composition and structure of native gellan gum produced by commercial fermentation is identical to the naturally occurring polysaccharide formed by Sphingomonas elodea on plants of Lily pad varieties.

Gellan Gums are usually water-soluble. Gellan gum is extremely effective at low use levels in forming gels, and are available in two types, high and low acyl content. Low acyl gellan gum products form firm, non-elastic, brittle gels, whereas high acyl gellan gum forms soft, very elastic, non-brittle gels. Varying the ratios of the two forms of gellan produces a wide variety of textures. The uniqueness of gellan gum is the ability to suspend while contributing minimal viscosity via the formation of a uniquely functioning fluid gel solution with a weak gel structure. Fluid gels exhibit an apparent yield stress, i.e., a finite stress which must be exceeded before the system will flow. These systems are very good at suspending particulate matter since, provided the stress exerted by the action of gravity on the particles is less than the yield stress, the suspension will remain stable. Other important properties of gellan gum fluid gels are the setting temperature, degree of structure and thermal stability. All of these properties are, as with normal unsheared gels, dependent upon the concentration of gellan gum and the type and concentration of gelling ions. This multi-functional hydrocolloid can be used at low levels in a wide variety of products that require gelling, texturizing, stabilizing, suspending, film-forming and structuring. Small size particles, low acyl gellan are especially suitable.

Maltodextrin gum is typically obtained by partial hydrolysis of starch. It is typically used in food products as a sweetening agent. It is usually found as a creamy-white hygroscopic spraydried powder. Maltodextrin is easily digestible, being absorbed as rapidly as glucose, and might be either moderately sweet or almost flavorless. It is commonly used for the production of natural sodas.

Maltodextrin consists of D-glucose units connected in chains of variable length. The glucose units are primarily linked with α(1→4) glycosidic bonds. Maltodextrin is typically composed of a mixture of chains that vary from three to nineteen glucose units long. Maltodextrins are typicallyclassified by DE (dextrose equivalent) and have typically a DE between 3 to 20. (The higher the DE value, the shorter the glucose chains, the higher the sweetness and the higher the solubility.) Above DE 20, the European Union's CN code calls it glucose syrup, at DE 10 or lower the ustoms CN code nomenclature classifies maltodextrins as dextrins.

Maltodextrin can be enzymatically derived from any starch. In the US, this starch is usually corn; in Europe, it is commonly wheat. While wheat-derived maltodextrin may cause concern for celiacs that it may contain gluten, maltodextrin is such a highly processed ingredient that the protein is removed, rendering it gluten free. If wheat is used to make maltodextrin, it will appear on the label. Even so, the maltodextrin will be gluten free.

Associations of gellan and xanthan or gellan and maltodextrin, or xanthan and maltodextrin or gellan and xanthan and maltodextrin are especially useful.

The amount of gum in the product can be for example ranging from 0.1 to 10% by weight. The amount can be for example ranging from 0.2 to 5% by weight, preferably of from 0.5 to 2%.

It is believed that the gum helps in providing viscosity to the product of the invention and mucosa protection against acid reflux in the esophagus. It is believed that the perception in the esophagus contributes to the perception of acidity relief. It is believed that the gum helps in providing viscosity to the product of the invention and mucosa protection against acid effects in the stomach.

### d) Phospholipid Product Such as Lecithin

The product of the invention comprises a phospholipid product. A phospholipid product is a phospholipid molecule or a composition comprising a phospholipid. The phospholipid product is for example lecithin. A phospholipid product can for example use phospholipid compositions or de-oiled phospholipid compositions.

A “phospholipid composition” as used herein is any composition containing a substantial proportion of phospholipids as defined below, e.g. at least 40 wt. % or in particular at least 50 wt. %. The phospholipid composition may further contain lipids other than phospholipids, including e.g. triglycerides, diglycerides, monoglycerides, fatty acids and glyco lipids, and carbohydrates. The weight percentages are expressed on a dry basis, i.e. in the assumed absence of water. A practical example of a phospholipid composition is lecithin.

A “de-oiled phospholipid composition” is a phospholipid composition having a reduced proportion of lipids, in particular triglycerides. The content of neutral lipids, i.e. triglycerides, diglycerides, monoglycerides and fatty acids, of the de-oiled phospholipid composition is less than 30 wt. %, more preferably less than 20 wt. %, most preferably less than 10 wt. %. The triglyceride content is preferably less than 20 wt. %, more preferably less than 10 wt. %, most preferably less than 5 wt. % of the de-oiled phospholipid composition. The phospholipid compositions may also contain carbohydrates, but preferably no more than 20 wt. %, in particular between 2 and 10 wt. % of the de-oiled phospholipids composition. Furthermore, the phospholipid composition may contain glyco lipids as defined below, e.g. between 1 and 50%, especially between 5 and 35 wt. % of the total de-oiled phospholipid composition. The content of phospholipids in the de-oiled phospholipid composition can be at least 65 wt. %, more preferably at least 75 wt. %, most preferably at least 80 wt. % of the de-oiled phospholipid composition. The proportion of phospholipids may also be lower, e.g. at least 45 wt. % or especially between 55 and 75 wt. %. If the composition also contains glyco lipids as defined below, the total proportion of phospholipids and glyco lipids taken together is preferably between 70 and 98 wt. %, more preferably between 80 and 95 wt. %. When disregarding any carbohydrates that may be present, the de-oiled phospholipids composition preferably contains at least 68 wt. % of phospholipids, more preferably at least 78 wt. % of phospholipids, if no glyco lipids are present.

In an alternative, the total proportion of phospholipids and glyco lipids taken together—disregarding any carbohydrates—is preferably between 73 and 99 wt. %, more preferably between 83 and 96 wt. %. In the latter case, the content of glyco lipids may be between 1 and 52, in particular between 5 and 37 wt. % of the de-oiled phospholipids composition (phospholipids, glycoplipids and neural lipids). The triglyceride content is preferably below 20 wt. %, more preferably below 10 wt. %, especially below 5 wt. %, and the combined neutral lipid content below, 31 wt. %, preferably below 20 wt. % and more preferably below 10 wt. % respectively.

For the purpose of the invention, a ‘phospholipid’ is any compound having a phosphate group and at least one long-chain hydrocarbon group, in particular a fatty acid residue (long-chain meaning at least 15 carbon atoms). In particular, a phospholipid contains one or two long-chain hydrocarbon groups on one side of the phosphate group, usually with an interconnecting glyceryl group, and optionally a polar group at another side of the phosphate group. The phospholipids preferably comprise one or more of phosphatidylcholine (PC), phosphatidylethanolamine (PE), phosphatidylserine (PS), phosphatidylinositol (PI), optionally phosphatidic acid (PA), phytoglycolipids and phosphosphingo lipids including sphingomyelin, i.e. phospholipids in which the diglyceride residue has been replaced by a ceramide unit, and which have a choline group, an inositol or other sugar residue or other polar group on the phosphate group. Also suitable are lyso-phospholipids (hydro lysed phospholipids), i.e. phospholipids having only one long-chain fatty acid residue per molecule. The phospholipids may be a single component, e.g. phosphatidylcholine, or, more commonly, a mixture of components.

A ‘glyco lipid’, which may be present in the (de-oiled) phospholipid composition, is understood to be a compound containing at least one long-chain hydrocarbon unit and at least one sugar unit which may also be sulphated (‘sulpholipid’). Suitable glyco lipids include glycosylated ceramides, such as GalCer, GIcCer, LacCer, and the more complex gangliosides, containing a chain of glycosyl residues, one or more which may carry a sialyl group. The phospholipid composition and de-oiled phospholipid composition to be used for coating the amino acids or peptides and/or proteins can be any food-grade phospholipid composition or lecithin.

Suitable phospholipids products that can be used include vegetal lecithin, egg lecithin, and marine lecithin. One can use for example oleaginous lecithin, such as soya lecithin, sunflower lecithin, rapeseed lecithin, egg lecithin, or fish oil lecithin.

The product of the invention can comprise for example from 0.1 to 10% by weight of phospholipid product, for example lecithin. This amount can preferably be ranging from 0.2 to 5%, more preferably from 0.5 to 1% by weight.

It is believed that phospholipids can prevent gastric irritation and protect the stomach. It is believed that phospholipids provide a protection of esophagus mucosa.

In some embodiments the phospholipid can be replaced or partially substituted with other edible food or pharmaceutical surfactants and/or emulsifiers.

### e) Base Product

The product of the invention comprises a base product. The base product comprises a mineral salt with a base anion. It is believed that the base anion acts as an acid neutralizer. According to the present invention, a base product can be typically considered as a product (compound or composition of matter) that provides to a composition in which it is introduced a pH increase of at least 0.1 pH units, preferably at least 0.2, more preferably at least 0.3, in comparison to the pH of a said composition without the base product. The pH of a liquid composition containing water can be measured directly. The pH of other compositions can be measured either after dilution at a 10% by weight concentration of the composition in water, or at a concentration in water such that the amount of base product is about 1.13% by weight, or at a concentration in water such that the amount of mineral salt of base anion is about 0.52% by weight.

Such base products are known by the one skilled in the art. Such products have been used in food product or in pharmaceutical products as antacid agents. The function of antacids is to neutralize the HCl secreted by gastric parietal cells by reaction with HCl to form chloride and water.

The mineral salt is preferably a sodium salt, a calcium salt, an aluminum salt, a magnesium salt, or a mixture thereof. The base product is for example a magnesium product, comprising a least a magnesium salt with a base anion.

The base anion can for example be selected from the group consisting of carbonate, hydroxide, oxide, salicilate, phosphate, sulfate, trisilicate, phosphate, organic acid anion, such as citrate, oxalate, ascorbate, salicilate, benzoate, phenylbutyrate or lactate, and mixtures or associations thereof. Preferred are carbonate, hydroxide, oxide, trisilicate, phosphate, organic acid anion, such as lactate, and mixtures thereof.

Examples of suitable salts include calcium caronate, calcium citrate, calcium hydroxide, calcium oxides, calcium phosphate, calcium phosphate, aluminum acetate, aluminum carbonate, alumunium hydroxide, aluminium oxide, aluminium phosphate, aluminum sulfate, hydrotalcites, magnesium carbonate, magnesium chloride, magnesium hydroxide, magnesium oxide, magnesium salicilate, magnesium phosphate, magnesium hydrogen phosphate, magnesium sulfate, magnesium trisilicate, magnesium salts of organic acids, such as magnesium lactate or citrate, sodium acetate, sodium bicarbonate, sodium bisphosphate, sodium borate, sodium carbonate, sodium citrate, dibasic sodium phosphate, sodium hydroxide, sodium hypochlorite, sodium hyposulfite, sodium lactate, sodium nitrite, sodium phenylbutyrate, sodium phosphate, sodium salicilate, sodium thiosulfate, and mixtures thereof. Preferred are magnesium hydroxide, magnesium oxide, magnesium trisilicate, magenisum phosphate, magnesium salts of organic acids such as magnesium lactate, and mixtures thereof.

The nature of the salt and its amount can be chosen in connection to the product form. For liquid product, the salt is preferably salt that is soluble in the matrix.

It is mentioned that most mineral salts of polymeric compounds are typically not considered as base products in the meaning of the invention. However, some gums salts can be considered (such as sodium alginate, sodium carboxymethyl cellulose). The salt can be different from a caseinate salt, an alginate salt, sodium citrate, a stearate salt, a carboxymethyl cellulose salt, calcium lactate, sodium phosphate, sodium pyrophosphate, calcium carbonate, sodium carbonate, calcium phosphate, sodium glutamate, an aspartate salt, magnesium bis aspartate, magnesium phosphate, magnesium hydrogen phosphate, calcium diphosphate, stearyllactate salts, sodium saccharin, calcium phosphate, aluminium hydroxide.

The nature and amount of the base product can be set to provide to the product of the invention a pH increase of at least 0.1 pH units, preferably at least 0.2, preferably at least 0.3, in comparison to the pH of the product without the base product.

In one embodiment, the base product is a magnesium product comprising:

optionally metallic magnesium, and
a magnesium salt preferably selected from the group consisting of magnesium carbonate, magnesium chloride, magnesium hydroxide, magnesium oxide, magnesium phosphate, magnesium salicilate, magnesium sulfate, magnesium trisilicate, magnesium salts of organic acids, such as magnesium lactate or citrate, and mixtures thereof. Preferred are magnesium hydroxide, magnesium oxide, magnesium trisilicate, magnesium phosphate, magnesium salts of organic acids such as magnesium lactate, and mixtures thereof.

The magnesium salt can comprise metallic magnesium. It is mentioned that the metallic magnesium might undergo salification in the composition product.

The base product, for example magnesium product, can comprise an aminoacid or an ammonium compound, for example glycine. This amino acid is linked by a covalent bond to the salt, preferably magnesium salt. It is believed that such embodiments allow improved solubility and/or improved bioavalability and/or improved organoleptic profiles. Examples and advantages of such embodiments are given in document EP 1949799. It is also believed that the bond between the mineral salt of the base product and an aminoacid such as glycine can break at the low pH of the stomach, therefore helping in delivering neutralizing properties.

It is mentioned that the base product can also include some further ingredients, for example ingredients that can help in handling and/or processing. Examples include maltodextrin, preservatives.

In a preferred embodiment, a magnesium product that is especially suitable for the invention comprises:

metallic magnesium,

MgO,

Mg Lactate, and

glycine.

Such products have a good solubility and provide good organoleptic properties to the product.

The amount of base salts is preferably ranging from 10 to 100% by weight of the base product. The amount of aminoacid or an ammonium compound can be typically ranging from 10 to 90% by weight of the base product. If a metal or mineral is present under a non salified form, its amount is preferably less than 50% by weight, more preferably less than 20%, and even more preferably less than 10% of the base product.

The product can comprise for example from 0.1 to 10% by weight of the base product, preferably from 0.2 to 5%, and more preferably from 0.5 to 3%. The product can comprise for example from 0.1 to 10% by weight of the mineral salt, preferably from 0.2 to 5%, preferably from 0.5 to 3%.

### f) Matrix

The product of the invention comprises a matrix.

In one embodiment, the product of the invention is a in a solid form. Such products are known by the one skilled in the art. Examples of such forms are a powder form, a tablet form, capsule form, or bar form. Such forms are known by the one skilled in the art. They can typically comprise a vector suitable for oral administration, for example excipients, such as binders, coatings, disintergrants, fillers, lubricants.

In one embodiment, the product of the invention is a product is in a liquid form. Such products are known by the one skilled in the art. In one embodiment, the matrix comprises animal milk, vegetal milk or fruit juice, or mixture thereof.

In one embodiment, the water content of the matrix is at least 50% by weight, for example from 50 to 99%.

The animal milk is typically cow milk, but one can use alternative animal milks such as sheep milk or goat milk. The vegetal milk can be for example soya milk.

In one embodiment the matrix comprises proteins. The matrix can comprise typically at least 1% by weight of proteins. Milks, either animal or vegetal typically comprise such proteins. Animal milk for example typically comprises casein.

The matrix can comprise microorganisms, such as lactic acid bacteria and/or probiotics (the probiotics can be lactic acid bacteria). Typical products comprising microorganisms are products such as milks or fruit juice with added microorganisms or fermented milk products, such as yogurts. Lactic acid bacteria are known by the one skilled in the art. Probiotics are also known by the one skilled in the art. Examples of probiotics include some Bifidobacteria and Lactobacilli, such as Bifidobacterium brevis, Lactobacillus acidophilus, lactobacillus casei, Bifidobacterium animalis, Bifidobacterium animalis lactis, Bifidobacterium infantis, bifidobacterium longum, lactobacillus casei, lactobacillus casei paracasei, lactobacillus reuteri, lactobacillus plantarum, lactobacillus rhamnosus.

In one embodiment the matrix is a fermented milk product such as a yogurt. It is mentioned that yogurts are considered, according to the invention, as being fermented milk products. It is believed that such matrix can participate in providing an anti-acid activity. That can typically provide a buffering effect of the pH, as well as the base product.

Fermented animal milk products are known by the one skilled in the art. Such products are essentially made up of animal milk, having undergone a fermentation step. The fermentation is typically done with microorganisms such as bacteria and/or yeast, preferably at least bacteria, and leads to the production of fermentation products, for example lactic acid and/or to multiplication of the microorganisms. The designation “fermented milk” can depend on local legislation, but is typically given to a dairy product prepared from skimmed or full fat milk, or else concentrated or powdered milk, having undergone a heat treatment at least equivalent to a pasteurization treatment, and inoculated with lactic acid producing micro-organisms such as lactobacilli (Lactobacillus acidophilus, Lb. casei, Lb. plantarum, Lb. reuteri, Lb. johnsonil), certains streptococci (Streptococcus thermophilus) bifidobacteria (Bifidobacterium bifidum, B. longum, B. breve, B. animalis) and lactococci (Lactococcus lactis). The matrix can be typically a yogurt product.

Fermented vegetal milk products are known by the one skilled in the art. Such product are products essentially made up vegetal milk, having a vegetal extract as a major constituent beyond water, having undergone a fermentation step. The fermentation is typically done with microorganisms such as bacteria and/or yeast, preferably at least bacteria, and leads to production of fermentation products, for example lactic acid and/or to multiplication of the microorganisms. By vegetal extract as a major constituent, it is typically referred to a vegetal content at least equal to 50% by weight of dry matter, preferably from 70% to 100%. The vegetal milk can be for example soya milk, oat milk, rice milk, almond milk, or a mixture thereof.

It is mentioned that the product of the invention can have a water content of at least 50% by weight, for example ranging from 50% to 99%. Water is typically present as part of the matrix.

The product of the invention can typically comprise from 0.1 to 99% by weight of the matrix, preferably from 50 to 99%, for example from 50% to 60%, or from 60% to 70%, or from 70% to 75%, or from 75% to 80%, or from 80% to 85%, or from 85% to 90%, or form 90% to 95%, or from 95% to 99%.

### Further Ingredients:

The product can comprise further ingredients. Such further ingredients might depend of the form and typical usage of the product. The further ingredients might be typical ingredients known in the field. Examples of further ingredients include:

sugar,

sweeteners such as aspartame and/or acesulfam and/or steviosides,

ferments,

vitamins,

nutrients additives, for example minerals,

thickeners,

preservatives,

stabilizers,

fibers,

flavors,

aroma,

fruits, nuts, extracts and mixture thereof.

### Further Features of the Product

In a preferred embodiment the product comprises a liquid matrix and is in a liquid form.

In a preferred embodiment the product comprises:

a) a salivation agent,
b) optionally a mouth and/or throat cooling agent,
c) at least one gum,
d) a phospholipid product,
e) a base product comprising a mineral salt with a base anion, preferably a magnesium, sodium, calcium, or aluminum salt, and
f) a liquid matrix.

In a preferred embodiment the product comprises:

a) a salivation agent,
b) optionally a mouth and/or throat cooling agent,
c) at least one gum,
d) a phospholipid product,
e) a base product comprising a mineral salt with a base anion, preferably a magnesium, sodium, calcium, or aluminum salt, and
f) a liquid matrix comprising milk, preferably animal milk, preferably a fermented milk.

In a preferred embodiment the product comprises:

a) a salivation agent,
b) a mouth and/or throat cooling agent,
c) at least one gum,
d) a phospholipid product,
e) a base product comprising a mineral salt with a base anion, preferably a magnesium, sodium, calcium, or aluminum salt, and
f) a liquid matrix.

In a preferred embodiment the product comprises:

a) a salivation agent,
b) a mouth and/or throat cooling agent,
c) at least one gum,
d) a phospholipid product,
e) a base product comprising a mineral salt with a base anion, preferably a magnesium, sodium, calcium, or aluminum salt, and
f) a liquid matrix comprising milk, preferably animal milk, preferably a fermented milk.

The product of the invention can comprise:

a) optionally from 0.0001% to 0.1% by weight of the salivation agent,
b) optionally from 0.0001% to 0.1% by weight of the mouth and/or throat cooling agent,
c) from 0.1 to 10% by weight of the gum,
d) from 0.1 to 10% by weight of phospholipid product,
e) from 0.1 to 10% by weight of the base product, and
f) from 0.1 to 99% by weight of matrix.

In a preferred embodiment the product comprises:

a) optionally from 0.0001% to 0.1% by weight of the salivation agent,
b) optionally from 0.0001% to 0.1% by weight of the mouth and/or throat cooling agent,
c) from 0.1 to 10% by weight of the gum,
d) from 0.1 to 10% by weight of phospholipid product,
e) from 0.1 to 10% by weight of the base product, and
f) from 0.1 to 99% by weight of liquid matrix.

In a preferred embodiment the product comprises:

a) optionally from 0.0001% to 0.1% by weight of the salivation agent,
b) optionally from 0.0001% to 0.1% by weight of the mouth and/or throat cooling agent,
c) from 0.1 to 10% by weight of the gum,
d) from 0.1 to 10% by weight of phospholipid product,
e) from 0.1 to 10% by weight of the base product, and
f) from 0.1 to 99% by weight of liquid matrix comprising milk, preferably animal milk, preferably a fermented milk.

In a preferred embodiment the product comprises:

a) from 0.0001% to 0.1% by weight of the salivation agent,
b) optionally from 0.0001% to 0.1% by weight of the mouth and/or throat cooling agent,
c) from 0.1 to 10% by weight of the gum,
d) from 0.1 to 10% by weight of phospholipid product,
e) from 0.1 to 10% by weight of the base product, and
f) from 0.1 to 99% by weight of liquid matrix.

In a preferred embodiment the product comprises:

a) from 0.0001% to 0.1% by weight of the salivation agent,
b) optionally from 0.0001% to 0.1% by weight of the mouth and/or throat cooling agent,
c) from 0.1 to 10% by weight of the gum,
d) from 0.1 to 10% by weight of phospholipid product,
e) from 0.1 to 10% by weight of the base product, and
f) from 0.1 to 99% by weight of liquid matrix, comprising milk, preferably animal milk, preferably a fermented milk.

In a preferred embodiment the product comprises:

a) from 0.0001% to 0.1% by weight of the salivation agent,
b) from 0.0001% to 0.1% by weight of the mouth and/or throat cooling agent,
c) from 0.1 to 10% by weight of the gum,
d) from 0.1 to 10% by weight of phospholipid product,
e) from 0.1 to 10% by weight of the base product, and
f) from 0.1 to 99% by weight of liquid matrix.

In a preferred embodiment the product comprises:

a) from 0.0001% to 0.1% by weight of the salivation agent,
b) from 0.0001% to 0.1% by weight of the mouth and/or throat cooling agent,
c) from 0.1 to 10% by weight of the gum,
d) from 0.1 to 10% by weight of phospholipid product,
e) from 0.1 to 10% by weight of the base product, and
f) from 0.1 to 99% by weight of liquid matrix, comprising milk, preferably animal milk, preferably a fermented milk.

It is mentioned that, in a particular embodiment, the product comprises the salivation agent a) and a magnesium product as base product e).

In some embodiments the product has a viscosity ranging from:

90 to 360 mPa·s at 10 s⁻¹ at 37° C., and/or

200 to 1000 mPa·s at 10 s⁻¹ at 10° C.

Such viscosities are believed to be especially suitable for liquid forms administrations. Such viscosities are believed to contribute to provide some relief from acidity sensation, and/or protection of the esophagus.

In some embodiments the product has a pH ranging from 4 to 8.5, preferably from 4 to 5. pHs ranging from 4 to 5 are preferred for products having a fermented matrix. It is believed that such pH and/or matrix can participate in providing an antacid activity and/or in providing a buffering effect. Products having non fermented matrixes, for example in solid forms, can have higher pHs, for example from 5 to 8.5.

### Process

The composition can be prepared by any appropriate process. The process involves mixing the various ingredients of the compositions. Accordingly the process can typically comprise the step of mixing optionally a), optionally b), c), d), e), and f) or components thereof. Typically some ingredients can be introduced as pre-mixes with other ingredients. Such processes are known by the one skilled in the art.

In a preferred process, the matrix f) is prepared, and the optionally a), optionally b), c), d) and e) are then introduced, as well as optional further ingredients.

The matrix f) can be a fermented milk product. Milk fermentation with lactic acid bacteria is known by the one skilled in the art. In an embodiment, the fermented milk is pasteurized after fermentation (typically at a temperature higher than 90° C., for example 100° C.).

The gum can be introduced in the matrix during the matrix preparation or in a further step, for example as a premix with other ingredients, for example in a syrup premix. In one embodiment, a part of the gum is introduced in the matrix during the matrix preparation step, and a part is introduced as a premix with other ingredients, for example in a syrup premix. Introduction of a part of gum in the matrix and a part in syrup allows an easier processing and viscosity control.

The base product, preferably a magnesium product, is preferably introduced into the matrix after the matrix preparation, especially if the matrix is a fermented matrix, as this ingredient might disturb the fermentation. The base product, preferably a magnesium product, can be introduced in a premix with other ingredients, for example in a syrup premix.

The syrup premix can typically comprise water, optionally sugar, whole or a part of gums, and the base product, preferably a magnesium product. The amount of water in the syrup can be of more than 50%, preferably more than 80%. The syrup and the matrix can typically be mixed and homogenized, typically in weight ratio syrup/matrix of 100/10 to 10/100, preferably of 100/25 to 50/50.

The phospholipid, the optional salivation agent, and the optional cooling agent, can be introduced in a fruit premix preparation, typically just before packaging the composition. The fruit preparation can typically further comprise a fruit extract, sugar, sweeteners, preservatives, texturing agents such as starch, and/or water. The weight ratio fruit preparation/rest of the formulation can be typically from 0.5/100 to 20/100, preferably from 2/100 to 8/100.

### Form, Packaging and Process of Use

The product of the invention is typically to be administered to humans. The administration is typically performed orally, for example as food or as a food supplement.

Depending on the form and packaging of the product, the product can be administered directly or after a preliminary preparation step, for example mixing with a fluid product such as water. This preliminary mixture is suitable, for example, for products in the form or powders, tablets, concentrated fluids in syrup or flasks forms.

For direct administration, the product can be packaged by appropriate means such as sealed cups, sealed bottles, capped cups, capped bottles. The volume of the package can vary. The package can accommodate a volume can be for example of from 10 mL to 50 mL, or from 50 mL to 100 mL, or from 100 mL to 150 mL, or of from 150 mL to 200 mL; or form 250 mL to 500 mL, or from 500 mL to 1000 mL, or over 1000 mL. Preferred volumes are typically of 50, 80, 100, 125, 150, 250, 500 or 1000 mL. In a preferred embodiment the volume corresponds to one serving. In a preferred embodiment an individual package corresponds to one serving of 50, 80, 100, 125 or 150 mL.

### Use—Effect

The product can be used in:

providing stomach and/or gastric comfort and/or health,

providing prevention of, relief from, and/or treatment from stomach and/or esophagus pain,

providing prevention of, relief from, and/or treatment of acidity or acidity sensation in the upper gastric sphere of humans, preferably in stomach and/or esophagus,

providing prevention of, relief to, and/or treatment of pyrosis and/or heartburn, and/or

providing prevention of, relief to, and/or treatment of Gastro-Esophageal Reflux Disease (GERD) and/dyspepsia.

In some embodiments the use involves protecting the esophagus of acid reflux by increasing the pH of the gastric liquid and/or by providing some covering of the esophagus mucosa. The gastric liquid refers to the content of the stomach. It is also referred to as stomach juice.

In one embodiment, the pH of the stomach juice is increased after administration of the composition of the invention, in comparison to the pH of the stomach juice before or without administration of the composition. In one embodiment the pH is increased of a mean value of less than 2.4 and/or the pH of stomach juice after administration of the composition is lower than 5. The pH of the stomach juice is preferably higher than 1.5 before administration of the composition.

The product of the invention can be used on a daily basis. It might be used for example on a daily basis during a period of more than 15 days, preferably more than 30 days, preferably without specific restrictions (for example on the package or on a notice). It is believed that the soft and consistent action of the product of the invention on the pH facilitates frequent and/or long-run uses.

The product can also be used occasionally upon acidity episodes.

Further details or advantages of the invention might appear in the following non-limiting examples.

### EXAMPLES

In the examples, the letter “C” indicates a comparative example.

### Example 1

### Composition 1

Composition 1 is prepared by mixing the following compositions, detailed below:

28.5 wt % of a fermented milk composition

66.5 wt % of a syrup composition

5 wt % of a fruit preparation

### Fermented Milk Composition

| | |
|---|---|
| Skimmed milk 0.05% GB | 96.16% |
| Skimmed milk in powder | 3.82% |
| Ferment | 0.01% |

Dry matter: 14%, water amount is about 90% of skimmed milk pH: 4.6

### Syrup Composition

| Water | to 100% |
|---|---|
| Sugar | 4.27% |
| Mixture of 33% Gellan* and 67% Sugar | 3.25% |
| Magnesium Salt | 1.7% |

*Gellan DL, Biotec

The magnesium salt is a mixture of 44.30% by weight of Mg Lactate, 1.50% by weight of Mg Oxide, 50.5% by weight of Glycine, 2.3% by weight of maltodextrin and 1.4% by weight of preservatives. In the mixture constituted of the 44.30% by weight of Mg Lactate and 1.50% by weight of Mg Oxide, there is a 6% by weight content of metallic magnesium.

### Fruit Preparation Composition

| Water | to 100% |
|---|---|
| Blend of fruits | 28.5000% |
| Sugar | 39.0000% |
| Soy Lecithin | 14.0000% |
| Flavors | 1.6000% |
| Starch | 2.0000% |
| Xanthan | 0.3000% |
| Sweeteners | 0.2800% |
| Ethyl Alcohol | 0.6998% |
| Cooling agent | 0.0358% |
| Salivating agent | 0.0870% |
| Potassium Sorbate | 0.1500% |

The cooling agent is a menthol based agent.
The salivating agent is a disodium succinate based agent.
The pH of composition 1 is 4.6. If the Mg salt is removed, the pH is 4.2.

### Example 2

### Compositions 2-9

Compositions similar to composition 1 are prepared, with some ingredients being removed.

L stands for Lecithin
G stands for Gellan
Mg stands for Mg salt
S stands for salivating agent

| Composition | 1 | 2C | 3C | 4C | 5C | 6C | 7C | 8C | 9C |
|---|---|---|---|---|---|---|---|---|---|
| Ingredient | / | S | L | G | Mg | L&G | L&Mg | L&Mg&G | Mg&G |
| removed | | | | | | | | | |
| Ingredient | All | L&G&Mg | G&Mg | L&Mg | L&G | Mg | G | None | L |
| present from | | | | | | | | | |
| L, G and Mg | | | | | | | | | |

### Example 3

### Rheology Evaluation

### Example 3.1

### Static Viscosity

The viscosity of the compositions is measured at a temperature of 10° C. and 37° C. Apparatus: Paar Physica MCR 300, PHYSICA Mefltechnik GmbH

Measuring system: DG26.7
Strain: from 1 s⁻¹ to 100 s⁻¹ and from 100 s⁻¹ to 1 s⁻¹.
Viscosity plotted at 10 s⁻¹.
The return curve is modelled following Oswald and Casson law depending on the samples.
The results of the viscosity plotted at 10 s⁻¹ are as follows:

| Composition | 1 | 3C | 4C | 5C | 6C | 7C | 8C | 9C |
|---|---|---|---|---|---|---|---|---|
| Ingredient | / | Lecithin | Gellan | Mg Salt | Lecithin | Lecithin | Lecithin | Mg Salt |
| removed | | | | | Gellan | Mg Salt | Mg Salt | Gellan |
| | | | | | | | Gellan | |
| Ingredient | All | G&Mg | L&Mg | L&G | Mg | G | None | L |
| present from | | | | | | | | |
| L, G and Mg | | | | | | | | |
| Viscosity at | 360 | 270 | 27 | 170 | 20 | 150 | 60 | 40 |
| 10° C. (mPa · s) | | | | | | | | |
| Viscosity at | 160 | 140 | 9 | 85 | 7 | 80 | 25 | 20 |
| 37° C. (mPa · s) | | | | | | | | |

Compared to composition 8C, the contribution of the ingredients to the viscosity is as follows:

| | | | Theoretical | Theoretical |
|---|---|---|---|---|
| | | | contribution to | contribution to |
| | Contribution to | Contribution to | viscosity at | viscosity at |
| | viscosity at | viscosity at | 10° C. (by | 37° C. (by |
| | 10° C. (from | 37° C. (from | adding | adding |
| Ingredient(s) | measurements) | measurements) | contributions) | contributions) |
| Mg salt | 20-60 = −40 | 7-25 = −18 | | |
| Lecithin | 40-60 = −20 | 20-25 = −5 | | |
| Gellan | 170-60 = 110 | 80-25 = 55 | | |
| Mg Salt + | 360-60 = 300 | 160-25 = 135 | 110-40-20 = 50 | 55-18-5 = 32 |
| Lecithin + | | | | |
| Gellan | | | | |

This shows that associating the gum, the base product and the phospholipid allows a viscosity synergy as the contribution of the association to the viscosity is higher than the sum of the individual contributions.

The return curves have been modelled following Oswald or Casson laws depending on the samples.

Samples containing the gum have clear pseudoplastic properties and the highest consistency coefficient K for the samples containing all the active ingredients. The composition comprising gellan have a pseudoplastic property (low n coefficient).

| | Consistancy coefficient | Behaviour coefficient |
|---|---|---|
| At 10° C. | (Oswald law), “K” (Pa · s) | Oswald law, n |
| Composition 1 | 1.473 | 0.358 |
| Composition 3C | 1.096 | 0.417 |
| Composition 5C | 0.833 | 0.335 |
| Composition 7C | 0.745 | 0.337 |

| | Consistancy coefficient | Behaviour coefficient |
|---|---|---|
| At 37° C. | (Oswald law), “K” (Pa · s) | Oswald law, n |
| Composition 1 | 0.599 | 0.454 |
| Composition 3C | 0.473 | 0.507 |
| Composition 5C | 0.318 | 0.437 |
| Composition 7C | 0.308 | 0.439 |

Samples without the gum have a low level of viscosity, and the yield stress can be considered as null.

| At 10° C. | Yield Stress (Pa) | Casson viscosity (mPa · s) |
|---|---|---|
| Composition 4C | 0.010 | 12.2 |
| Composition 6C | 0.006 | 11.0 |
| Composition 8C | 0.34 | 7.9 |
| Composition 9C | 0.06 | 9.2 |

| At 37° C. | Yield Stress (Pa) | Casson viscosity (mPa · s) |
|---|---|---|
| Composition 4C | 0.01 | 3.5 |
| Composition 6C | 0.006 | 3.3 |
| Composition 8C | 0.34 | 2.9 |
| Composition 9C | 0.06 | 3.3 |

### 3.2 Dynamic Viscosity

The viscosity of the products have been measured for different strains during 1 min at a temperature of 10° C. and 37° C. to measure time depends and recovering capacities of the product.

### Apparatus: Paar Physica MCR 300, PHYSICA Mefltechnik GmbH

Measuring system: DG26.7
Strain: 1 minute at 10 s⁻¹, then 1 minute at 50 s⁻¹, then 1 minute at 10 s¹

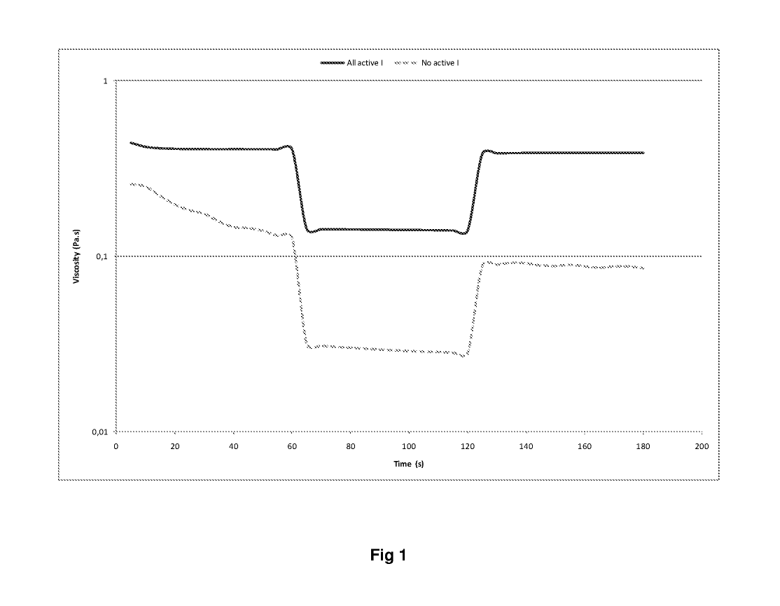

The results are show one FIG. 1, where “All active” stands for composition 1, and “No active” stands for composition 8C)

FIG. 1 represents the plot of viscosity as a function of strain (reported as time) for a product according to the invention and a comparative product.

The viscosity of the compositions of the invention stays constant during time at a given shear rate, when for the matrix composition 8C the viscosity decrease with time (thixotropy). The viscosity to the compositions of the invention are pseudoplastic (viscosity depends on shear rate). Moreover the product recovers quickly its viscosity upon reduction of shear.

### Example 4

### In Vitro Stomach Acidity Tests

The impact of compositions on the pH of stomach juice has been evaluated in vitro. During a day the volume of the stomach can vary from large range of volumes. The study measures the pH after addition of the different samples to two different volumes of a reconstituted stomach juice at pH 1.8, which is a low pH, corresponding to an acidity having a bad impact upon reflux in the oesophagus.

### Stomach Juice Preparation

1) Preparing a Stomach electrolyte (10× Stock-1000 ml)

Weigh in a volumetric flask of 11:
31.0 g NaCl
11.0 g KCl
1.50 g CaCl₂H₂O

Add ca 800 ml demineralised water and a stirring magnet. Solubilize everything by stirring and flush the neck of the flask with demiwater to get all in solution. When everything is solved, remove magnet and fill the flask up to mark.

2) Preparing stomach juice (500 ml)

Pipet 50 ml Stomach electrolyte (10× stock) in a 0.51 volumetric flask.

Add ca 400 ml demineralised water and a stirring magnet.

Add 7.5 ml 1 M NaHCO₃

While stirring titrate pH to 4.0 with 1 M HCl.

Remove magnet and fill the flask to mark.

Pour solution in a 1 liter schott bottle, add stirring magnet and put on ice.

When T<7° C. add while stirring:
70 mg pepsin (porcine stomach, sigma p7012) weighed on paper, and
gradually, to prevent lumps from forming, 93.75 mg lipase (Rhizopus oryzae, DF 15K Amano Pharmaceutical Co, Ltd Nagoya)

Keep solution on ice.

The pH of the stomach juice is equal to 4.

### Protocol

The stomach juice is realized as mentioned above but without the enzyme addition.

Stomach juice volume: 50 mL and 300 mL

Stomach juice to pH 1.8 with a 1 M HCl solution.

For each stomach juice volume add 3 servings (80 mL) for each composition tested.

Follow the pH after addition of each serving for the each starting stomach juice volume.

Results are presented below:

| | Number of | | Composition 5C |
|---|---|---|---|
| | servings | Composition 1 | (without Mg salt) |
| pH measured | 0 | 1.8 | 1.8 |
| after servings | 1 | 2.98 | 2.34 |
| on a 300 mL | 2 | 3.65 | 3.08 |
| stomach volume | 3 | 3.85 | 3.43 |
| pH measured | 0 | 1.8 | 1.8 |
| after servings | 1 | 4.35 | 3.8 |
| on a 50 mL | 2 | 4.4 | 4 |
| stomach volume | 3 | 4.6 | 4.05 |

The results confirm that the composition efficiently in addresses the acidity of the stomach.

The presence of the base product allows an improvement in re-establishing a higher pH. The improvement is of about 20% for 300 ml of stomach juice. The improvement is of about 15% for 50 mL of stomach juice.

### Example 5

### Comparison with Other Acidity-Relievers Products

The effect on pH of the stomach of compositions 1 and some commercial products is evaluated. The evaluation is carried out at various starting pH, for various stomach volume, after several servings.

Commercial products tested:

| Commercial | | | |
|---|---|---|---|
| Product | | Reported active | |
| name | Source | ingredient | Posology |
| Rennie | Marketed in | hydrotalcite 1 g | 1 liquid sachet (can |
| Liquo ® | France, 2010 | (90 mg of | be renewed after 2 |
| | | aluminium) | hours) |
| Rennie ® | Marketed in | Calcium carbonate | 1 to 2 tablet |
| | France, 2010 | (680 mg) | Can be renewed to |
| | | Magnesium | 4-8 a day |
| | | carbonate (80 mg) | |
| Maalox ® | Marketed in | aluminium oxide | 1 to 2 liquid sachet |
| | France, 2010 | 230 mg (hydrated | Can be renewed to |
| | | aluminum oxide | 6 a day |
| | | 460 mg) | |
| | | Magnesium | |
| | | hydroxide 400 mg | |
| Gaviscon ® | Marketed in | Sodium Alginate | 1 to 2 liquid sachet |
| | France, 2010 | (500 mg) | Can be renewed to |
| | | Sodium bicarbonate | 6 a day |
| | | (267 mg) | |
| | | Calcium Carbonate | |
| | | (no amount) | |

### Protocol

The stomach juice is realized as mentioned above but without the enzyme addition.

Stomach juice volume: 50 mL and 300 mL

Stomach juice to pH 1.8 or 3 with a 1 M HCl solution.

For each stomach juice volume add up to 3 servings of commercial product or composition 1 (1 serving of composition 1=80 mL).

Follow the pH after addition of each serving for each starting volume of stomach juice.

The final pH is reported below

| | Stomach | Number | Rennie | | | | |
|---|---|---|---|---|---|---|---|
| Starting pH | juice volume | of serving | Liquo | Rennie | Maalox | Gaviscon | Composition 1 |
| 1.8 | 50 | 1 | 5.6 | 7.3 | 5.1 | 6.9 | 4.35 |
| 1.8 | 50 | 2 | | 8 | 6.42 | 7.23 | 4.4 |
| 1.8 | 50 | 3 | | | | | 4.6 |
| 1.8 | 300 | 1 | 4.7 | 5.6 | 3.1 | 3.5 | 2.98 |
| 1.8 | 300 | 2 | | 6.2 | 5.5 | 6.1 | 3.65 |
| 1.8 | 300 | 3 | | | | | 3.85 |
| 3 | 50 | 1 | 6 | 6.7 | 5.6 | 7.1 | 4.54 |
| 3 | 50 | 2 | | 7 | 6.1 | 7.3 | 4.56 |
| 3 | 50 | 3 | | | | | 4.6 |
| 3 | 300 | 1 | 5.4 | 6.1 | 3.1 | 6.1 | 4.5 |
| 3 | 300 | 2 | | 6.45 | 5.5 | 6.55 | 4.54 |
| 3 | 300 | 3 | | | | | 4.55 |
| Mean | | | 5.4 | 6.66 | 5 | 6.34 | 4.26 |
| final pH | | | | | | | |
| Standard | | | 0.544 | 0.757 | 1.270 | 1.244 | 0.507 |
| deviation | | | | | | | |
| Mean | | | 3 | 4.26 | 2.6 | 3.95 | 1.86 |
| increase | | | | | | | |

Whatever the starting conditions tested (stomach pH and volume), the composition has a softer effect on final pH than the commercial products (mean pH value=4.26 vs from 5 to 6.6). Furthermore, the capacity to control the final stomach pH is better with the composition as the standard deviation is lower than for all others products.

Moreover, considering that the mean starting pH (1.8+3)/2=2.4, the increase (mean final pH minus mean starting pH) is of 1.86 pH units for composition 1, which is below 2.4.

Moreover the final pH is always of lower than 4.7.

A too high stomach pH during a long period of time might cause digestion problem by enzyme inactivation. It can also decrease absorption level of certain vitamins and minerals and some medications. The composition is more adapted to a regular consumption to control acidity sensation than commercial products.

### Example 6

### In Vitro Saliva Induction Test—pH of Alimentary Bolus

The impact of saliva induction on pH of alimentary bolus and on buffering capacity is simulated by adding different volumes of artificial saliva to compositions to be tested.

### Protocol:

1) Prepare the artificial saliva:

Weight: 6.2 g NaCI, 2.2 g KCI, 0.3 g CaCl₂.2H₂O, and 1.2 g NaHCO₃,

Add ca 800 ml demineralised water and a stirring magnet. Solubilise everything by stirring and flush the neck of the flask with demineralised water to get all in solution. When everything is solubilised, titrate pH to 6.3 with 1 and 0.1 M HCl, remove magnet and fill the flask up to mark.

Refrigerate.

Add 72 mg alpha-amylase (Sigma A 6211) per 100 ml

2) Simulate saliva digestion of the compositions (mouth digestion) for various saliva volumes:

Mix 80 ml of composition 1 and 4, 6, 8, 10, 12.5 or 15 mL of saliva and allow incubation 5 minutes at 37° C. This simulates alimentary bolus with various characteristics (Volume, pH).

3) Measure pH of alimentary bolus, and volume of 1 m HCl needed to bring the pH to 3. Results are reported below

### Results for Saliva Digestion

| | Saliva volume added to 80 mL composition | | | | | |
|---|---|---|---|---|---|---|
| | 4 | 6 | 8 | 10 | 12.5 | 15 |
| pH | 4.44 | 4.44 | 4.44 | 4.435 | 4.47 | 4.47 |
| Volume needed for | 24.2 | 24.2 | 24.2 | 26.85 | 28.6 | 28 |
| having pH = 3 | | | | | | |

For a small amount of saliva, the pH of the composition is not impacted. For a higher volume (typically over 10 ml) of saliva the pH increases. As normal people have a flow rate of saliva of 1 mLJ min (15 mL for 15 min), people under the normal have a flow about 0.5 to 0.7 mL/min(7.5 mL to 10.5 mL for 15 min) and people with a too small salivation production have a flow lower than 0.5 mL/min, the presence of a salivation agent allows increasing the volume of saliva typically over 10 ml and increasing the pH regulation.

### Example 7

### In-Vitro Impact of Alimentary Bolus on Stomach pH

The aim of this in vitro test is to test two different aspects of the digestion:

firstly the impact of each sample on the pH of the alimentary bolus (saliva plus samples) and the volume of HCl needed to reach pH=3

Secondly the impact of the enzyme on the pH and viscosity.

### Protocol:

### 1) Prepare the Artificial Saliva:

Weight: 6.2 g NaCI, 2.2 g KCI, 0.3 g CaCl₂.2H₂O, and 1.2 g NaHCO₃,

Add ca 800 ml demineralised water and a stirring magnet. Solubilise everything by stirring and flush the neck of the flask with demineralised water to get all in solution. When everything is solubilised, titrate pH to 6.3 with 1 and 0.1 M HCl, remove magnet and fill the flask up to mark.

Refrigerate.

Add 72 mg alpha-amylase (Sigma A 6211) per 100 ml

2) Simulate saliva digestion of the compositions (mouth digestion) for various saliva volumes:

Mix 80 ml of composition 12.5 mL of saliva and allow incubation 5 minutes at 37° C. This simulates an alimentary bolus.

3) Measure pH of alimentary bolus, add 1M HCl needed to bring the alimentary bolus pH to 3, and measure the volume needed. Results are reported below (as “pH 1”).
4) Add to alimentary bolus (at pH 3) to 25 ml of stomach juice (preparation described above) and incubate for 1 hour at 37° C. Measure the pH and measure the viscosity. Results are reported below (as “pH 2”).

### Alimentary Bolus Results

| Composition | 1 | 8C |
|---|---|---|
| Ingredient | / | L&Mg&G |
| removed | | |
| Ingredient | All | None |
| present from | | |
| L, G and Mg | | |
| pH 1 | 4.435 | 4.175 |
| Volume | 26.85 | 12.45 |
| pH 2 | About 3 | About 3 |

This shows that the base product has an impact on the pH of the alimentary bolus.

After stomach digestion by enzymes bile action, there is no increase of the pH (final pH is of about 3 in all cases). The viscosity has also been measured and did not show significant differences after digestion. This shows that the agents used are well digested and do not cause adverse effects in digestion.

### Example 8

### Panel Tests

Composition 1 was evaluated by a trained test panel subjected to acidity episodes. A test panel, with a consumption of 2 products a day over 2 weeks, was performed. The population sample was composed by 243 men and women, from 25 to 65 years old, with sporadic or frequent acidity. They were all non dairy rejecter's people.

More than 75% thought that the product helped them to feel better
More than 85% felt de diminution of acidity episodes
More than 90% found the product refreshing
More than 50% found the product more effective than usual acidity relievers.

This shows that the product of the invention provides interalia stomach and/or gastric comfort and/or health, as well as refreshment.

### Example 9

### Buffering Test

### Protocol

One measures the quantity of acid needed to reach pH 2. One adds 0.2 mL to 1 mL of a 0.5M HCl solution to 80 mL of a sample.
From this pH measurement in function of base or acid added, one calculates the buffering. The samples tested are:

Composition 1 according to the invention,

Water: Volvic® (containing a low amount of minerals)

Milk: a skimmed milk pasteurized.

A standard yogurt

### Results

| Sample | Water | Milk | Yogurt | Composition 1 |
|---|---|---|---|---|
| Volume of HCl | 4.8 | 30.6 | 32.6 | 40.6 |
| solution | | | | |
| needed (ml) | | | | |

| | |
|---|---|
| Variation | 538% |

6.5%

24.5%

This shows that the composition according to the example has much higher buffering power than any of the other samples.

### Example 10

### Salivation Test

A salivation blind test comparing composition 1 and a comparative product identical to composition 1 without the salivating agent is performed on a population of 24 participants.

### Protocol

Participants have to drink the 80 mL in as low sips as possible in order to avoid salivation swallow. The samples are tested on two different days but at the same moment of the day, 10 h30. Wth a questionnaire an evaluation of the food intake before the test is performed. The aim is to evaluate if the consumption is the same to avoid artifact that might be linked to increase water consumption for example. The saliva is collected in cups, pre-weighted, during 5 minutes. Participants have to spit whenever they needed over the 5 min. This time is chosen to give the time to molecules to induce salivation and to have enough volume of saliva to perceive differences. People have to mention the number of sips they needed to drink the product. The quantity of saliva in the cups is then weighted to evaluate salivation.

14 of 24 participants salivate more with composition 1 than with the comparative product.
This shows that products comprising a salivation agent are effective when combined with gum c) phospholipids d) and base product e) in a liquid matrix.

1. A product, comprising:
a) optionally a salivation agent,
b) optionally a mouth and/or throat cooling agent,
c) at least one gum,
d) a phospholipid product,
e) a base product comprising a mineral salt with a base anion, preferably a magnesium, sodium, calcium, or aluminum salt, and
f) a solid or liquid matrix.

2. The product according to claim 1, wherein the product is a food product, a food supplement product or a pharmaceutical product.

3. The product according to claim 1, wherein the salivation agent is selected from the group consisting of mono-, di-, or tricarboxylic acids or salts thereof, ascorbic acid and succinic acid or a salt thereof.

4. (canceled)

5. The product according to claim 1, wherein the cooling agent is a menthol compound, a cyclic carboxamide compound, or a mixture thereof.

6. (canceled)

7. The product according to claim 1, wherein the gum is selected from the group consisting of the following compounds: Gellan gums, xanthan gums, maltlodextrins, locust bean gums, carraghenan gums, alginates, carboxymethylcelluloses, methylcelluloses, and mixtures thereof.

8. The product according to claim 1, wherein the phospholipid product is lecithinselected from the group consisting of soy lecithin, sunflower lecithin, rapeseed lecithin and fish oil lecithin.

9. The product according to claim 1, wherein the base product is a magnesium product, comprising at least a magnesium salt with a base anion selected from the group consisting of carbonate, hydroxide, oxide, trisilicate, phosphate, organic acid anion, such as lactate, and mixtures thereof.

10. (canceled)

11. The product according to claim 1, wherein the base product is a magnesium product comprising: metallic magnesium or a magnesium salt selected from the group consisting of magnesium carbonate, magnesium hydroxide, magnesium oxide, magnesium trisilicate, magnesium phosphate, magnesium salts of organic acids, such as magnesium lactate, and mixtures thereof.

12. (canceled)

13. The product according to claim 11, wherein the magnesium product comprises metallic magnesium, MgO, Mg Lactate, and glycine.

14. The product according to claim 1, wherein the matrix is a liquid comprising animal milk, vegetal milk or fruit juice.

15. The product according to claim 1, wherein the matrix comprises at least 1% by weight of proteins.

16. The product according to claim 1, wherein the product has a water content ranging from 50 to 99% by weight.

17. The product according to claim 14, wherein the matrix comprises microorganisms selected from the group consisting of lactic acid bacteria and probiotics.

18. (canceled)

19. The product according to claim 14, that is a yogurt or a fermented milk product.

20. The product according to claim 1, that is in a powder form, a tablet form, a capsule form, or a bar form.

21. The product according to claim 1 comprising:
a) optionally from 0.0001% to 0.1% by weight of the salivation agent,
b) optionally from 0.0001% to 0.1% by weight of the mouth and/or throat cooling agent,
c) from 0.1 to 10% by weight of the gum
d) from 0.1 to 10% by weight of phospholipid product,
e) from 0.1 to 10% by weight of the base product, and
f) from 0.1 to 99% by weight of matrix.

22. The product according to claim 1 having a viscosity ranging from 90 to 360 mPa·s at 10 s″1 at 37° C., and/or 200 to 1000 mPa·s at 10 s″1 at 10° C.

23. The product according to claim 1, having a pH ranging from 4 to 8.5.

24. A process of making the product according to claim 1, comprising the step of mixing at least one gum, a phospholipid product, a base product comprising a mineral salt with a base anion, preferably a magnesium, sodium, calcium, or aluminum salt, and a solid or liquid matrix optionally in combination with a salivation agent and/or a mouth and/or throat cooling agent or components thereof.

25. A method of providing prevention of or relief from and/or treatment of stomach or esophagus pain in a subject in need thereof comprising administering the product of claim 1 to the subject.

26. A method for protecting the esophagus of a subject from acid reflux, comprising administering the product of claim 1 to the subject in an amount effective to increase the pH of the gastric liquid and/or providing some covering of the mucosa of the esophagus.

27. The method according to claim 26, wherein the pH of stomach juice after administration of the product is increased, compared to the pH of stomach juice before or without administration of product.

28. The method according to claim 27, where the pH is increased of a mean value of less than 2.4 and/or the pH of stomach juice after administration of the composition is less than 5.

29. The method according claim 26, for daily use, during a period of more than 15 days.
